(19) United States
(12) Patent Application Publication
(10) Pub. No.: US 2023/0050359 A1
(43) Pub. Date: Feb. 16, 2023

(54) ASSISTIVE APPARATUS FOR SINGLE PORT SURGICAL ROBOT

(71) Applicant: INDUSTRY-ACADEMIC COOPERATION FOUNDATION, YONSEI UNIVERSITY, Seoul (KR)

(72) Inventor: Sangwun KIM, Seoul (KR)

(73) Assignee: INDUSTRY-ACADEMIC COOPERATION FOUNDATION, YONSEI UNIVERSITY, Seoul (KR)

(21) Appl. No.: 17/780,181

(22) PCT Filed: Aug. 11, 2020
(86) PCT No.: PCT/KR2020/010579

§ 371 Date: May 26, 2022

(30) Foreign Application Priority Data
Sep. 3, 2019 (KR) 10-2019-0108694

(51) Int. Cl. A61B 17/02; A61B 17/34

(52) U.S. Cl. CPC A61B 17/02; A61B 17/3423; A61B 34/30

(57) ABSTRACT

The present invention relates to an assistive apparatus for a single port surgical robot, including a single port housing which forms a chamber communicating with a wound retractor and is coupled to the wound retractor with airtightness maintained, and a plurality of guide tubes which are provided in the single port housing to communicate with the chamber and form passages through which a surgical tool mounted on an arm of the single port surgical robot and assistive surgical tools for assisting with single port surgery are selectively inserted, wherein the plurality of guide tubes have different heights and are provided to protrude from the single port housing.

### TECHNICAL FIELD

The present invention relates to an assistive apparatus for a single port surgical robot, and more particularly, to an assistive apparatus for a single port surgical robot which secures a surgical angle of each surgical tool while preventing the surgical tools from colliding with each other, and enables stable surgery when the surgery is performed without using a trocar in a state in which the surgical tools mounted in a single port surgical robot arm and assistive surgical tools for assisting with single port surgery are simultaneously inserted into a single port coupled to a wound retractor.

### BACKGROUND ART

Recently, unlike open surgery which is performed by opening a large incision in the abdomen of a patient, many forms of minimally invasive surgery have been attempted as surgical methods that minimize the size of an affected area.

Unlike open surgery, such minimally invasive surgery has advantages of less postoperative pain, quick recovery of intestinal motility, early intake of food, a short hospitalization period, a quick return to a normal state, and an excellent cosmetic effect because the incision range is narrow. Due to these advantages, minimally invasive surgery is being used for cholecystectomy, stone removal, prostate cancer surgery, hernia correction, and the like, and the field is gradually expanding.

Meanwhile, laparoscopic surgery is widely known as a type of minimally invasive surgery.

Laparoscopic surgery is a method in which holes with sizes of 0.5 to 1.5 cm are drilled in the abdomen of a patient, a camera and various surgical tools are inserted through the holes, and then surgery is performed while the inside of the abdominal cavity is observed.

For laparoscopic surgery, a plurality of holes are drilled in the abdomen, a trocar is inserted into each of the holes, surgical tools such as an endoscope, a gas injector, a laser generator, and a cutter are inserted into the trocars one-to-one, and then the surgery is performed while the inside is observed.

That is, the conventional trocar has a structure in which only one surgical tool is inserted through one trocar.

Therefore, in order to perform the laparoscopic surgery using a conventional trocar to which surgical tools are coupled one-to-one, it is necessary to drill a number of holes in the patient's abdomen. This not only damages the patient's skin accordingly, but also uses a large number of trocars, which incurs a cost burden on the patient.

In order to reduce this problem, so-called ‘single port laparoscopic surgery’ has become widely known in recent years among laparoscopic surgery methods, and single port surgery is a method in which a single hole, that is, a ‘single port,’ is drilled in the patient's abdomen, and all surgical tools are inserted through the single port.

Single port laparoscopic surgery can minimize scars as compared to conventional laparoscopic surgery, but has a disadvantage in that the surgical method is not easy.

Therefore, in recent years, in order to compensate for the disadvantage of single port laparoscopic surgery, robot surgery using a surgical robot to perform single port laparoscopic surgery has been spotlighted as an alternative.

However, a single port housing for a single port surgical robot has not been developed yet, and therefore a single port housing for laparoscopy is being used instead. The single port housing for a robot requires a large port (27 mm), but the single port housing for laparoscopy does not have a large port to insert a robotic arm. However, according to a US Patent Publication, a single port housing for a robot has been registered, but is not being sold yet. Since the single port housing for a single port surgical robot is flat, and a plurality of guide tubes have the same height as the single port housing, when surgery is performed by inserting surgical tools mounted in the single port surgical robot arm and assistive surgical tools for assisting with the single port surgery into the guide tubes of the single port housing at the same time, there are problems that, since housings surrounding the outside of each of the surgical tools collide with each other, it is difficult to secure a surgical angle of the surgical tool, and since it is difficult to secure surgery space for the surgical tools in the abdominal wall in which the surgical tools are operated, the surgery is not easy.

### TECHNICAL PROBLEM

The present invention is directed to providing an assistive apparatus for a single port surgical robot, in which, when surgery is performed by inserting surgical tools mounted in a single port surgical robot arm and assistive surgical tools for assisting with single port surgery into guide tubes of a single port housing at the same time, housings surrounding the outside of each of the surgical tools are prevented from colliding with each other, a surgical angle of each of the surgical tools can be secured, it is possible to secure surgery space for the surgical tools in the abdominal wall in which the surgery is performed, and the surgery can be stably and easily performed.

### TECHNICAL SOLUTION

One aspect of the present invention provides an assistive apparatus for a single port surgical robot, including a single port housing which forms a chamber communicating with a wound retractor and is coupled to the wound retractor with airtightness maintained, and a plurality of guide tubes which are provided in the single port housing to communicate with the chamber and form passages through which a surgical tool mounted on an arm of the single port surgical robot and assistive surgical tools for assisting with single port surgery are selectively inserted, wherein the plurality of guide tubes have different heights and are provided to protrude from the single port housing.

Here, the assistive apparatus for a single port surgical robot may further includes entry caps mounted on the guide tubes to form entrance holes through which the surgical tool mounted on the arm of the single port surgical robot inserted into the guide tube and the assistive surgical tools for assisting with single port surgery enter and exit.

The plurality of guide tubes may have different diameters, and outer diameters of the entry caps may have sizes larger than outer diameters of the guide tubes.

The entry caps may be formed of a stretchable material.

The assistive apparatus for a single port surgical robot may further include a check valve which is selectively provided in each of the guide tubes to open the passage when the surgical tool mounted on the arm of the single port surgical robot and the assistive surgical tools for assisting with single port surgery are inserted into the chamber through the guide tubes and to close the passage when the surgical tool mounted on the arm of the single port surgical robot and the assistive surgical tools for assisting with single port surgery are separated from the chamber and prevents air in the chamber from being discharged through the guide tubes to an outside.

The assistive apparatus for a single port surgical robot may further include a first valve member which is provided in the single port housing to communicate with the chamber and injects a gas into a human body, and a second valve member which is provided in the single port housing to communicate with the chamber and discharges the gas inside the human body to an outside.

### ADVANTAGEOUS EFFECTS

According to the present invention, when surgery is performed by inserting surgical tools mounted in a single port surgical robot arm and assistive surgical tools for assisting with single port surgery into guide tubes of a single port housing at the same time, housings surrounding the outside of each of the surgical tools are prevented from colliding with each other, a surgical angle of each of the surgical tools can be secured, it is possible to secure surgery space for the surgical tools in the abdominal wall in which the surgery is performed, and the surgery can be stably and easily performed.

### DESCRIPTION OF DRAWINGS

FIG. 1 is a perspective view of an auxiliary device for a single port surgical robot according to an embodiment of the present invention.

FIG. 2 is a front view of FIG. 1.

FIG. 3 is a plan view of FIG. 1.

FIG. 4 is a cross-sectional view along line A-A of FIG. 1.

### MODES OF THE INVENTION

In order to achieve the above object, the present invention is achieved by an assistive apparatus for a single port surgical robot including a single port housing which forms a chamber communicating with a wound retractor and is coupled to the wound retractor with airtightness maintained; and a plurality of guide tubes which are provided in a single port housing to communicate with the chamber and form a passage through which surgical tools mounted in a single port surgical robot arm and assistive surgical tools for assisting with single port surgery are selectively inserted, wherein the plurality of guide tubes have different heights and are provided to protrude from the single port housing.

Advantages and features of the present invention and methods of achieving them will become apparent with reference to embodiments described below in detail in conjunction with the accompanying drawings. However, the present invention is not limited to the embodiments disclosed below and may be implemented in various different forms, and the present embodiments are provided only to complete the disclosure of the present invention and to fully inform those of ordinary skill in the art to which the present invention pertains of the scope of the present invention.

The terminology used herein is for the purpose of describing particular embodiments only and is not intended to be limiting of the invention. As used herein, singular forms are intended to include plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof. It will be understood that, although the terms “first,” “second,” etc. may be used herein to describe various elements, these elements should not be limited by these terms. These terms are only used to distinguish one element from another. For example, a first element could be termed a second element, and, similarly, a second element could be termed a first element, without departing from the scope of the present invention.

Unless otherwise defined, all terms (including technical and scientific terms) used herein have the same meanings as commonly understood by those of ordinary skill in the art to which this invention belongs. It will be further understood that terms such as those defined in commonly used dictionaries should be interpreted as having meanings consistent with their meanings in the context of the relevant art and will not be interpreted in an idealized or overly formal sense unless expressly so defined herein.

Hereinafter, the present invention will be described in detail with reference to the accompanying drawings.

Prior to the description, although it is described in the present embodiment that an assistive apparatus for a single port surgical robot according to the present invention is applied to laparoscopic surgery, it is stated in advance that the present invention is not limited thereto and can be applied to single port surgery using a da Vinci surgical robot or the like that is applied to various surgical operations such as thoracoscopic surgery, in addition to laparoscopic surgery.

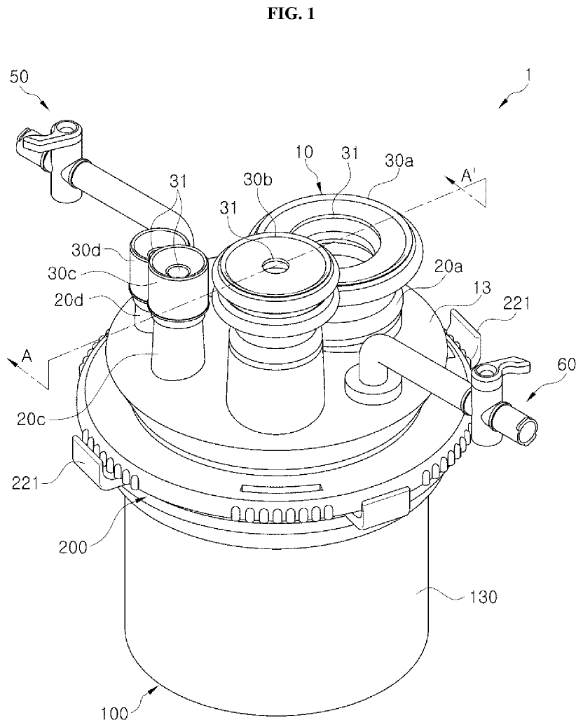

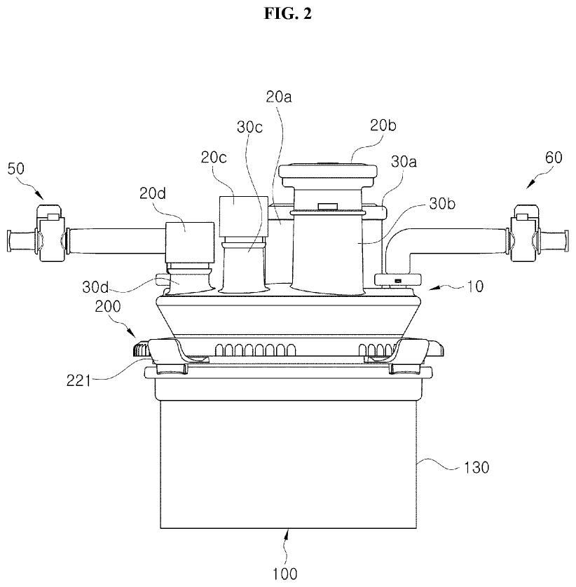

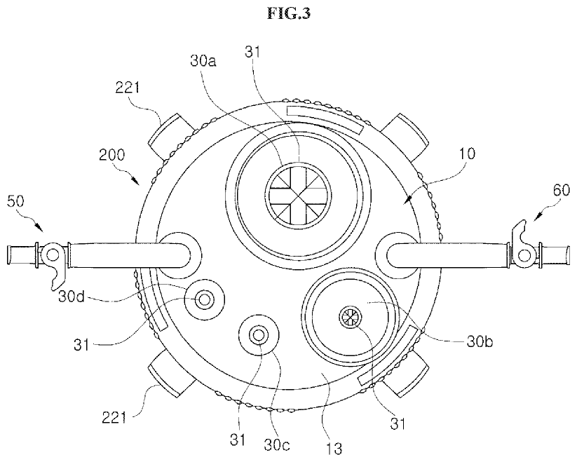

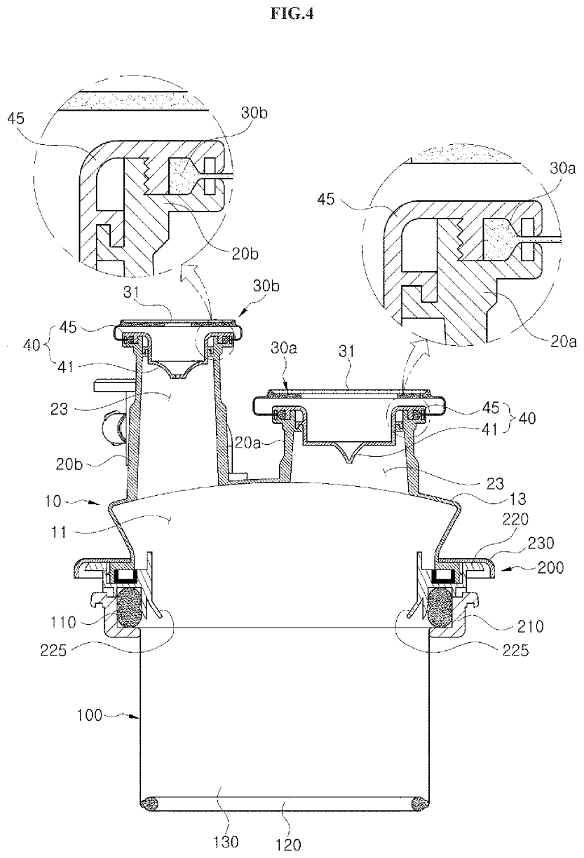

FIGS. 1 to 4 illustrate an assistive apparatus for a single port surgical robot according to an embodiment of the present invention.

As illustrated in the drawings, the assistive apparatus 1 for a single port surgical robot according to the embodiment of the present invention includes a single port housing 10.

The single port housing 10 is coupled to a wound retractor 100 with airtightness maintained.

Here, in order to help with understanding of the present invention, a configuration of the wound retractor 100 to which the assistive apparatus 1 for a single port surgical robot according to the present invention is coupled will be briefly described.

The wound retractor 100 is inserted into a patient's abdominal incision during surgery and widens the incision not only to guide a surgical tool mounted in an arm of the single port surgical robot and assistive surgical tools (hereinafter referred to as ‘surgical tool’ for convenience of description) for assisting with single port surgery into the human body, but also to support each of the surgical tools, and also serves to prevent infection due to extracted parts by preventing the extracted parts from coming into contact with the incision during surgery.

The wound retractor 100 includes a first fixing ring 110, a second fixing ring 120, and a sleeve 130.

The first fixing ring 110 has a ring shape and is disposed outside a human body. In the present embodiment, the first fixing ring 110 has a shape of a double ring unlike the second fixing ring 120, but the first fixing ring 110 may have the same ring shape as the second fixing ring 120.

The second fixing ring 120 has a ring shape and is disposed apart from the first fixing ring 110. The second fixing ring 120 is inserted into the human body, and an original ring shape thereof is restored to form a passage so that a plurality of surgical tools can be inserted into the human body.

The sleeve 130 has a hollow cylindrical shape and flexibly connects the first fixing ring 110 to the second fixing ring 120. The sleeve 130 prevents the surgical tool from coming in direct contact with the incision of the skin and widens the field of view for the surgical tool maximally.

Meanwhile, the first fixing ring 110 and the second fixing ring 120 have elasticity such that the first fixing ring 110 and the second fixing ring 120 are deformed when the first fixing ring 110 and the second fixing ring 120 receive an external force and return to their original shapes when the external force is removed. The first fixing ring 110 and the second fixing ring 120 include silicone, urethane, or a synthetic resin. Thus, an operator can bend or fold the second fixing ring 120 and then can insert the second fixing ring 120 into a human body through an incision such as patient's navel. The sleeve 130 may be formed of a film-type stretchable synthetic resin, for example, silicone or urethane.

After the second fixing ring 120 is inserted into an incision site, as the original shape thereof is restored, a passage is formed so that a plurality of surgical tools can be inserted into the human body, the second fixing ring 120 serves to fix the sleeve 130 on a back surface of the skin so that the sleeve 130 does not come out of the human body during surgery, and since the sleeve 130 is in close contact with the back surface of the skin, an injected gas is prevented from leaking through a space between the back surface of the skin and the sleeve 130.

Further, the first fixing ring 110 may adjust a length of the sleeve 130 by adjusting a winding degree of the sleeve 130 on the first fixing ring 110 to fit a skin thickness of the patient.

Returning to the main point again, the single port housing 10 of the assistive apparatus 1 for a single port surgical robot according to the embodiment of the present invention has a cylindrical shape of which a lower side is open.

A lower opening of the single port housing 10 is coupled to the first fixing ring 110 of the wound retractor 100 by a connector 200 with airtightness maintained.

The connector 200 includes a first fixing ring accommodation member 210, a detachable member 220, and a cover 230.

The first fixing ring accommodation member 210 forms a step along the inner side thereof, and the first fixing ring of the wound retractor 100 is accommodated in a step region.

The detachable member 220 is attached to or detached from an upper end portion of the first fixing ring accommodation member 210 with the first fixing ring 110 interposed therebetween. A distal end portion of the detachable member 220 may include a plurality of lugs 221 coupled to the upper end portion of the first fixing ring accommodation member 210. Meanwhile, a protector 225 inclined in an inward direction of the sleeve 130 may be provided inside the detachable member 220 in order to prevent the surgical tool inserted into the wound retractor 100 through the single port housing 10 from coming into contact with the sleeve 130 of the wound retractor 100 and giving an impact.

The cover 230 may be formed in a vertically separable structure, and the cover 230 is coupled to an upper surface of the detachable member 220 around the opening of the single port housing 10.

In order for the chamber 11 of the single port housing 10 to have a sealed structure, the cover 230 and the detachable member 220 must be sealed. To this end, the perimeter of the opening of the single port housing 10 extends toward a lower portion of the cover 230 and is disposed between the cover 230 and the detachable member 220 to seal the chamber 11 of the single port housing 10.

Meanwhile, the single port housing 10 of the assistive apparatus 1 for a single port surgical robot according to the embodiment of the present invention has a cylindrical shape of which a lower side is open, and the chamber 11 communicating with the wound retractor is formed therein.

Further, the assistive apparatus 1 for a single port surgical robot according to the embodiment of the present invention includes the plurality of guide tubes 20a, 20b, 20c, and 20d.

The plurality of guide tubes 20a, 20b, 20c, and 20d are provided in the single port housing 10 to communicate with the chamber 11.

The plurality of guide tubes 20a, 20b, 20c, and 20d are provided to protrude at intervals from an end portion opposite to the lower opening of the single port housing 10, for example, from an upper plate 13 of the single port housing 10. The plurality of guide tubes 20a, 20b, 20c, and 20d are disposed at intervals in consideration of a limited area of the upper plate of the single port housing 10 and a size of the surgical tool mounted on the arm of the surgical robot.

Each of the guide tubes 20a, 20b, 20c, and 20d has a cannula shape, the passage 23 through which the surgical tool mounted on the arm of the surgical robot is inserted is formed therein, and the passage 23 communicates with the chamber 11.

In addition, the plurality of guide tubes 20a, 20b, 20c, and 20d of the assistive apparatus 1 for a single port surgical robot according to the embodiment of the present invention have different heights. Also, the plurality of guide tubes 20a, 20b, 20c, and 20d have different diameters. That is, guide tubes having different sizes may be provided according to the type of surgical tool. Here, although not shown, as another embodiment, some of the plurality of guide tubes may have the same height and the same diameter. In addition, the diameters and positions of the guide tubes may be variously changed according to surgery.

Meanwhile, the surgical tool mounted on the arm of the single port surgical robot can be inserted through a first guide tube 20a having a relatively large diameter among the plurality of guide tubes, and the assistive surgical tools for assisting with single port surgery may be selectively inserted through the remaining guide tubes 20b, 20c, and 20d.

In this way, since the plurality of guide tubes 20a, 20b, 20c, and 20d protrude from the single port housing 10 at different heights, when single port surgery is performed using a surgical robot, for example, when the surgery is performed in a state in which surgical tools mounted on the arm of the surgical robot, for example, treatment devices such as a laparoscopic forceps for holding tissue during surgery, endoscopes for observing the inside of the abdominal cavity, laser generators for direct treatment of affected areas, and ultrasound generators, are inserted through the first guide tube 20a of the single port housing 10, and assistive surgical tools for holding neighboring organs, withdrawing tissues and extracted parts, or performing hemostasis are simultaneously inserted through the remaining guide tubes 20b, 20c, and 20d, collision between housings (not shown) surrounding the outside of the surgical tools is prevented. Further, when the surgical tool mounted on the arm of the single port surgical robot enters the abdomen, it opens, the surgical angles of the remaining assistive surgical tools are smoothly secured based on the surgical tool mounted on the arm for the surgical robot, it is possible to secure an operating space of the surgical tools in the abdominal wall in which surgery is performed, and an operator can move freely and can operate stably and easily during the surgery.

Further, the assistive apparatus 1 for a single port surgical robot according to the embodiment of the present invention may further include entry caps 30a, 30b, 30c, and 30d.

The entry caps 30a, 30b, 30c, and 30d form entrance holes 31 through which the surgical tools inserted into the guide tubes 20a, 20b, 20c, and 20d enter and exit, and are mounted on free ends of the guide tubes 20a, 20b, 20c, and 20d exposed to the outside.

Outer diameters of the entry caps 30a, 30b, 30c, and 30d may be larger than or equal to outer diameters of the guide tubes 20a, 20b, 20c, and 20d. Also, the entry caps 30a, 30b, 30c, and 30d may be fitted around the guide tubes 20a, 20b, 20c, and 20d.

The entry caps 30a, 30b, 30c, and 30d seal the outer periphery of each of the surgical tools that are inserted into the chamber 11 of the single port housing 10 through the entrance holes 31 and block air outflow in the chamber 11. The entry caps 30a, 30b, 30c, and 30d may include a stretchable material, for example, a silicone, urethane, or rubber material that is elastically deformed by an external force and is restored to an original shape thereof when the external force is removed.

In this way, since the entry caps 30a, 30b, 30c, and 30d include a stretchable material, it is possible to block the air outflow in the chamber 11 by sealing the outer periphery of the surgical tool when the surgical tool enters and exists. Further, when the single port surgery is performed in a state in which the surgical tool mounted on the arm of the single port surgical robot and the assistive surgical tools for assisting with the single port surgery are simultaneously inserted into the guide tubes 20a, 20b, 20c, and 20d of the single port housing 10, if necessary, the surgical angles of the surgical tools, particularly, the surgical angles of the assistive surgical tools, are secured smoothly based on the surgical tool mounted on the surgical robot arm, and it is possible to secure the operating space of the surgical tools in the abdominal wall in which surgery is performed, and an operator can move freely and operate stably and easily during the surgery.

In addition, the assistive apparatus 1 for a single port surgical robot according to the embodiment of the present invention may further include a check valve 40.

The check valve 40 is provided on the inside of each of the guide tubes 20a, 20b, 20c, and 20d to seal the outer periphery of the surgical tool. The check valve 40 includes a valve body 41 and a valve support 45.

The valve body 41 has a membrane shape having elasticity which opens when an external force is applied in one direction to open and close the passage 23 of each of the guide tubes 20a, 20b, 20c, and 20d and contracts when the external force is removed in one direction. For example, the check valve 40 opens the passage 23 of each of the guide tubes 20a, 20b, 20c, 20d when the surgical tool is inserted into the chamber 11 through the guide tubes 20a, 20b, 20c, and 20d and closes the passage 23 of each of the guide tubes 20a, 20b, 20c, and 20d when the surgical tool is separated from the chamber 11. Thus, it is possible to prevent the air in the chamber 11 from being discharged to the outside through the guide tubes 20a, 20b, 20c, and 20d.

The valve support 45 supports one end portion of the valve body 41 and is screwed to the guide tubes 20a, 20b, 20c, and 20d. Thus, the check valve 40 is supported by each of the guide tubes 20a, 20b, 20c, and 20d.

On the other hand, the entry caps 30a, 30b, 30c, and 30d are closely supported along the outer periphery of the valve support 45 screwed to each of the guide tubes 20a, 20b, 20c, and 20d, and firmly support the check valve 40 on the inside of each of the guide tubes 20a, 20b, 20c, and 20d. Thus, the check valve 40 is not separated from each of the guide tubes 20a, 20b, 20c, and 20d.

Here, the check valve 40 may be selectively provided in the guide tubes 20a, 20b, 20c, and 20d.

In addition, the assistive apparatus 1 for a single port surgical robot according to the embodiment of the present invention may further include a first valve member 50 and a second valve member 60.

The first valve member 50 and the second valve member 60 are provided in the single port housing 10 to independently communicate with the chamber 11.

The first valve member 50 forms a gas injection path through which a gas for securing a surgical space for a surgical site inside the human body is injected, and opens and closes a gas flow path.

The second valve member 60 forms a gas discharge path for discharging the gas injected into the human body and the gas discharged from the surgical site to the outside, and opens and closes a gas discharge path.

With such a configuration, after the assistive apparatus 1 for a single port surgical robot according to the embodiment of the present invention is mounted on the wound retractor 100 inserted into a hole of the abdomen, the surgical tool mounted on the arm of the single port surgery robot and the assistive surgical tools for assisting with the single port surgery are inserted through the corresponding entry caps 30a, 30b, 30c, and 30d and guide tubes 20a, 20b, 20c, and 20d and are inserted into an affected area through the wound retractor 100, and thus surgery can be performed.

At this time, since the plurality of guide tubes 20a, 20b, 20c, and 20d through which each of the surgical tools is inserted have different heights from each other, when single port surgery is performed using a single port surgical robot, the housings surrounding the outside of the surgical tool mounted on the arm of the surgical robot and the assistive surgical tools for assisting with single port surgery do not collide with each other, the surgical angles of the assistive surgical tools are secured smoothly based on the surgical tool mounted on the ram of the surgical robot, the operating space of the surgical tools can be secured in the abdominal wall in which the surgery is performed, and also the operator can freely move and can perform the surgery stably and easily.

Although an embodiment of the present invention has been above described with reference to the accompanying drawings, those skilled in the art to which the present invention pertains will be able to understand that the present invention may be embodied in other specific forms without changing the technical spirit or essential features thereof. Therefore, it should be understood that the embodiments described above are illustrative in all respects and not restrictive.

1. An assistive apparatus for a single port surgical robot, the apparatus comprising:
a single port housing which forms a chamber communicating with a wound retractor and is coupled to the wound retractor with airtightness maintained; and
a plurality of guide tubes which are provided in the single port housing to communicate with the chamber and form passages through which a surgical tool mounted on an arm of the single port surgical robot and assistive surgical tools for assisting with single port surgery are selectively inserted,
wherein the plurality of guide tubes have different heights and are provided to protrude from the single port housing.

2. The assistive apparatus of claim 1, further comprising entry caps mounted on the guide tubes to form entrance holes through which the surgical tool mounted on the arm of the single port surgical robot inserted into the guide tube and the assistive surgical tools for assisting with single port surgery enter and exit.

3. The assistive apparatus of claim 2, wherein the plurality of guide tubes have different diameters, and outer diameters of the entry caps have sizes larger than outer diameters of the guide tubes.

4. The assistive apparatus of claim 2, wherein the entry caps are formed of a stretchable material.

5. The assistive apparatus of claim 1, further comprising a check valve which is selectively provided in each of the guide tubes to open the passage when the surgical tool mounted on the arm of the single port surgical robot and the assistive surgical tools for assisting with single port surgery are inserted into the chamber through the guide tubes and to close the passage when the surgical tool mounted on the arm of the single port surgical robot and the assistive surgical tools for assisting with single port surgery are separated from the chamber and prevents air in the chamber from being discharged through the guide tubes to an outside.

6. The assistive apparatus of claim 1, further comprising a first valve member which is provided in the single port housing to communicate with the chamber and injects a gas into a human body; and a second valve member which is provided in the single port housing to communicate with the chamber and discharges the gas inside the human body to an outside.
